(19) United States
(12) Patent Application Publication
(10) Pub. No.: US 2024/0242837 A1
(43) Pub. Date: Jul. 18, 2024

(54) MACHINE LEARNING METHODS TO PREDICT MENOPAUSE SYMPTOMS AND TREATMENT OPTIONS

(71) Applicant: ASTELLAS PHARMA INC., Tokyo (JP)

(72) Inventors: Jin Lee, Northbrook, IL (US); Chad Dau, Northbrook, IL (US); Alexander Chettiath, Northbrook, IL (US); Ritu Saxena, Northbrook, IL (US)

(21) Appl. No.: 18/563,354

(22) PCT Filed: May 20, 2022
(86) PCT No.: PCT/IB22/54754

§ 371 Date: Nov. 21, 2023

Related U.S. Application Data
(60) Provisional application No. 63/191,751, filed on May 21, 2021.

(51) Int. Cl. G16H 50/20; G16H 10/20; G16H 10/60; G16H 50/70

(52) U.S. Cl. CPC G16H 50/20; G16H 10/20; G16H 10/60; G16H 50/70

(57) ABSTRACT

Techniques are provided for training machine learning models to predict menopause outcome trajectories and effective menopause interventions treatments for patients based on patient parameters. An example method includes obtaining historical EMR data associated with a plurality of historical patients, analyzing the historical EMR data associated with the plurality of historical patients to determine one or more patient parameters associated with each historical patient and a menopause outcome trajectory associated with each historical patient, generating training datasets that include patient parameters and menopause outcome trajectories associated with each historical patient and training a menopause outcome machine learning model, using the training datasets, to predict a menopause outcome trajectory and/or the effectiveness of a menopause intervention or treatment for a given patient based on the patient's patient parameters.

### CROSS-REFERENCE TO RELATED APPLICATIONS

The present application claims priority to U.S. Provisional Application No. 63/191,751, filed May 21, 2021, entitled “Machine Learning methods to Predict Menopause Symptoms and Treatment Options,” the entire disclosure of which is incorporated by reference herein.

### FIELD OF THE DISCLOSURE

The technology of this disclosure pertains generally to medical diagnostic methods or systems, and more particularly to machine learning based methods or systems that can accurately identify the symptoms associated with menopause, assess stage of menopause and provide a menopause outcome trajectory.

### BACKGROUND

Generally speaking, menopause occurs when a woman stops having menses for twelve consecutive months and can no longer become pregnant naturally. Menopause is preceded by perimenopause, during which a woman's menstrual periods may become irregular before stopping completely. Menopause usually begins between the ages of 45 and 55, but can develop before or after this age range. There are 34 symptoms of menopause, including: irregular periods, hot flashes, vaginal dryness, night sweats, mood swings, bloating, sore breasts, weight gain, changes in libido, headaches, pain in joints, electric shock sensations, burning tongue, gum problems, digestive issues, dry and itchy skin, anxiety, tingling sensation in the extremities, sleeping difficulties, difficulty concentrating, dizziness, fatigue, loss of hair, memory lapse, brittle nails, tight muscles, stress incontinence, changes in body odor, irritability, allergies, irregular heartbeat, depression, panic disorder, and osteoporosis.

Surveys show that 80% of women age 40-65 experience menopausal symptoms and other issues related to menopause, but only 25% of women age 40-65 receive adequate care for these symptoms and other issues. Moreover, further surveys show that 84% of women between ages 50-59 say that menopausal symptoms interfere with their lives, and 12% of women say that these symptoms interfere with their lives “a great deal” or are otherwise debilitating.

However, in the same surveys, 42% of women between ages 50-59 say that they have never discussed menopause with a healthcare provider. In fact, only one in five women between ages 50-59 said that they had received a referral to a menopause specialist. Moreover, only 20% of OB-GYN residencies teach menopause, and to the extent that menopause is taught in an OB-GYN residency, it is taught as an elective. Furthermore, surveys show that 80% of medical residents feel “barely comfortable” discussing or treating menopause in patients. Consequently, many women are uninformed about menopause and the symptoms they may experience during menopause.

### SUMMARY

The present disclosure provides systems and methods for training a machine learning model to predict a menopause outcome trajectory for patients based on patient parameters. Generally speaking, historical electronic medical record (EMR) data associated with historical patients may be analyzed (e.g., using natural language processing techniques) to identify characteristics of patients before, during, and after menopause. While EMR data is discussed generally herein, in some examples, the EMR data may be supplemented with and/or replaced by additional medical data associated with historical patients from other sources, such as, e.g., transcription data from healthcare providers associated with historical patients, results of imaging data associated with historical patients, genetic or other biomarker data associated with historical patients, statistical data from research papers or articles associated with historical patients, survey data associated historical patients (e.g., including patient-reported outcome data), biometric data associated with historical patients (e.g., physical activity data, heart rate data, step count, sleep data, etc., from fitness trackers or other mobile devices associated with patients), laboratory testing data (such as the outcomes of laboratory tests prescribed to patients by health care professionals including one or more of: estradiol laboratory test, uric acid laboratory test, testosterone laboratory test, Hba1c laboratory test, presence or absence of abnormal hormone level tests, FSH laboratory test, or estrogen level laboratory test), and/or other patient-provided data associated with historical patients.

In any case, the medical data associated with the historical patients may be analyzed to determine various demographic, medical, and lifestyle data associated with each historical patient, as well as data indicating a historical menopause outcome trajectory information for each historical patient. The historical menopause outcome trajectory information for a historical patient may include, e.g., indications of ages at which perimenopause, menopause, and/or post-menopause began for each historical patient, which specific symptoms each historical patient experienced and at what age, the severity of each symptom, etc. Additionally, in some examples, the medical data associated with the historical patients may be analyzed to identify various interventions that were used for preventing and/or alleviating menopause symptoms experienced by historical patients and their effectiveness for those historical patients. The results of this data analysis may be used as a training dataset, to train a machine learning model to generate a predicted menopause outcome trajectory for a given patient based on demographic, medical, and lifestyle data associated with the patient. In some examples, this data analysis may be further used to create a training dataset to train a machine learning model to generate predicted interventions that may be successful for preventing and/or alleviating predicted menopause symptoms for a given patient based on demographic, medical, and lifestyle data associated with the patient.

The trained machine learning models may be implemented in a software application, and patients, healthcare providers, or other users may provide demographic, medical, and/or lifestyle data associated with a given patient as input, e.g., via a user interface of the software application, and receive a predicted menopause outcome trajectory for the patient as an output, e.g., via the user interface of the software application. For instance, in some examples, the predicted menopause outcome trajectory may include an indication of ages at which perimenopause, menopause, and/or post-menopause are predicted to begin for the patient, as well as symptoms that the patient is predicted to experience at various ages, and the predicted severity of those symptoms. Moreover, in some examples, the predicted menopause outcome trajectory may include an indication of literature or other resources that may be helpful in understanding various symptoms predicted to be experienced by the patient, and/or an indication of one or more interventions that are predicted to be successful for preventing and/or alleviating various symptoms predicted to be experienced by the patient. For instance, these interventions may include medications, recommended lifestyle changes, or other recommendations, based on the symptoms that the patient is predicted to experience.

Advantageously, using the systems and method provided herein, patients and healthcare providers may be provided with a personalized predicted menopause outcome trajectory that indicates predicted ages at which the patient may expect to experience perimenopause, menopause, and/or post-menopause, and various symptoms related thereto, along with, in some cases, resources that explain the process and possible treatments for alleviating any predicted symptoms. Accordingly, patients may be better informed regarding what they should expect as they approach perimenopause and menopause, and may consequently be better able to seek out treatments for their perimenopause and menopause symptoms.

In an aspect, a computer-implemented method is provided, comprising: obtaining, by one or more processors, historical electronic medical record (EMR) data associated with a plurality of historical patients; analyzing, by the one or more processors, the historical EMR data associated with the plurality of historical patients to determine one or more patient parameters associated with each historical patient and a historical menopause outcome trajectory associated with each historical patient; generating, by the one or more processors, a training dataset that includes patient parameters and historical menopause outcome trajectories associated with each historical patient; and training, by the one or more processors, a menopause outcome machine learning model, using the training dataset, to generate a predicted menopause outcome trajectory for a patient based on patient parameters associated with the patient, wherein the predicted menopause outcome trajectory includes or consists of predictions of one or more of: an age of onset of perimenopause for the patient, an age of onset of menopause for the patient, an age of onset of post-menopause for the patient, particular menopause symptoms experienced by the patient, an age of onset of each menopause symptom experienced by the patient, or a severity level associated with each menopause symptom experienced by the patient.

Preferably, the predicted menopause outcome trajectory includes or consists of predictions of: (i) one or more of an age of onset of perimenopause for the patient, an age of onset of menopause for the patient, and an age of onset of post-menopause; (ii) one or more, two or more, 3 or more, 4 or more, 5 or more of particular menopause symptoms expected be experienced by the patient, such as number or frequency of hot flashes, sleep disruption, fatigue, sexual dysfunction, vaginal dryness, presence of menstruation, characteristics of menstruation, instances of amenorrhea, night sweats, urinary symptoms, mood symptoms, brain fog, changes in ability to focus, hair loss, weight gain, depression, anxiety, dizziness, vertigo, inflammation, and/or central nervous system conditions, possibly together with the severity level associated with some or each/all of these symptoms.

In another aspect, a computer system is provided, comprising: one or more processors; and a non-transitory program memory communicatively coupled to the one or more processors and storing executable instructions that, when executed by the one or more processors, cause the processors to: obtain historical electronic medical record (EMR) data associated with a plurality of historical patients; analyze the historical EMR data associated with the plurality of historical patients to determine one or more patient parameters associated with each historical patient and a historical menopause outcome trajectory associated with each historical patient; generate a training dataset that includes patient parameters and historical menopause outcome trajectories associated with each historical patient; and train a menopause outcome machine learning model, using the training dataset, to generate a predicted menopause outcome trajectory for a patient based on patient parameters associated with the patient, wherein the predicted menopause outcome trajectory includes or consists of predictions of one or more of: an age of onset of perimenopause for the patient, an age of onset of menopause for the patient, an age of onset of post-menopause for the patient, particular menopause symptoms experienced by the patient, an age of onset of each menopause symptom experienced by the patient, or a severity level associated with each menopause symptom experienced by the patient.

In still another aspect, a non-transitory computer readable storage medium storing computer-readable instructions is provided. The computer-readable instructions, when executed by one or more processors, cause the one or more processors to: obtain historical electronic medical record (EMR) data associated with a plurality of historical patients; analyze the historical EMR data associated with the plurality of historical patients to determine one or more patient parameters associated with each historical patient and a historical menopause outcome trajectory associated with each historical patient; generate a training dataset that includes patient parameters and historical menopause outcome trajectories associated with each historical patient; and train a menopause outcome machine learning model, using the training dataset, to generate a predicted menopause outcome trajectory for a patient based on patient parameters associated with the patient, wherein the predicted menopause outcome trajectory includes or consists of predictions of one or more of: an age of onset of perimenopause for the patient, an age of onset of menopause for the patient, an age of onset of post-menopause for the patient, particular menopause symptoms experienced by the patient, an age of onset of each menopause symptom experienced by the patient, or a severity level associated with each menopause symptom experienced by the patient.

In an additional aspect, a computer-implemented method is provided, comprising: obtaining, by one or more processors, patient parameters associated with a patient; analyzing, by the one or more processors, the patient parameters associated with the patient using a trained menopause outcome machine learning model; and generating, by the one or more processors, using the trained menopause outcome machine learning model, a predicted menopause outcome trajectory for the patient based on the patient parameters associated with the patient, wherein the menopause outcome trajectory includes or consists of one or more of: an age of onset of perimenopause for the patient, an age of onset of menopause for the patient, an age of onset of post-menopause for the patient, particular menopause symptoms experienced by the patient, an age of onset of each menopause symptom experienced by the patient, or a severity level associated with each menopause symptom experienced by the patient; and wherein the menopause outcome machine learning model is trained by: obtaining historical electronic medical record (EMR) data associated with a plurality of historical patients; analyzing the historical EMR data associated with the plurality of historical patients to determine one or more historical patient parameters associated with each historical patient and a historical menopause outcome trajectory associated with each historical patient; generating a training dataset that includes historical patient parameters and historical menopause outcome trajectories associated with each historical patient; and training the menopause outcome machine learning model, using the training dataset, to generate a predicted menopause outcome trajectory for new patients based on new patient parameters.

In another aspect, a computer system is provided, comprising: one or more processors; and a non-transitory program memory communicatively coupled to the one or more processors and storing executable instructions that, when executed by the one or more processors, cause the processors to: obtain patient parameters associated with a patient; analyze the patient parameters associated with the patient using a trained menopause outcome machine learning model; and generate, using the trained menopause outcome machine learning model, a predicted menopause outcome trajectory for the patient based on the patient parameters associated with the patient, wherein the menopause outcome trajectory includes or consists of one or more of: an age of onset of perimenopause for the patient, an age of onset of menopause for the patient, an age of onset of post-menopause for the patient, particular menopause symptoms experienced by the patient, an age of onset of each menopause symptom experienced by the patient, or a severity level associated with each menopause symptom experienced by the patient; and wherein the menopause outcome machine learning model is trained by: obtaining historical electronic medical record (EMR) data associated with a plurality of historical patients; analyzing the historical EMR data associated with the plurality of historical patients to determine one or more historical patient parameters associated with each historical patient and a historical menopause outcome trajectory associated with each historical patient; generating a training dataset that includes historical patient parameters and historical menopause outcome trajectories associated with each historical patient; and training the menopause outcome machine learning model, using the training dataset, to generate a predicted menopause outcome trajectory for new patients based on new patient parameters.

In still another aspect, a non-transitory computer readable storage medium is provided, storing computer-readable instructions that, when executed by one or more processors, cause the one or more processors to: obtain patient parameters associated with a patient; analyze the patient parameters associated with the patient using a trained menopause outcome machine learning model; and generate, using the trained menopause outcome machine learning model, a predicted menopause outcome trajectory for the patient based on the patient parameters associated with the patient, wherein the menopause outcome trajectory includes or consists of one or more of: an age of onset of perimenopause for the patient, an age of onset of menopause for the patient, an age of onset of post-menopause for the patient, particular menopause symptoms experienced by the patient, an age of onset of each menopause symptom experienced by the patient, or a severity level associated with each menopause symptom experienced by the patient; and wherein the menopause outcome machine learning model is trained by: obtaining historical electronic medical record (EMR) data associated with a plurality of historical patients; analyzing the historical EMR data associated with the plurality of historical patients to determine one or more historical patient parameters associated with each historical patient and a historical menopause outcome trajectory associated with each historical patient; generating a training dataset that includes historical patient parameters and historical menopause outcome trajectories associated with each historical patient; and training the menopause outcome machine learning model, using the training dataset, to generate a predicted menopause outcome trajectory for new patients based on new patient parameters.

### BRIEF DESCRIPTION OF THE DRAWINGS

FIG. 1 illustrates a block diagram of an example system for training a machine learning model to predict a menopause outcome trajectory for patients based on patient parameters, in accordance with some examples.

FIG. 2 illustrates a schematic diagram of example inputs and outputs used to train a machine learning model to predict a menopause outcome trajectory for patients based on patient parameters, in accordance with some examples described herein.

FIGS. 3A-3D illustrate example user interface displays for a user menopause application, in accordance with some examples.

FIG. 4 illustrates a flow diagram of an example method for training a machine learning model to predict a menopause outcome trajectory for patients based on patient parameters, in accordance with some examples.

### DETAILED DESCRIPTION

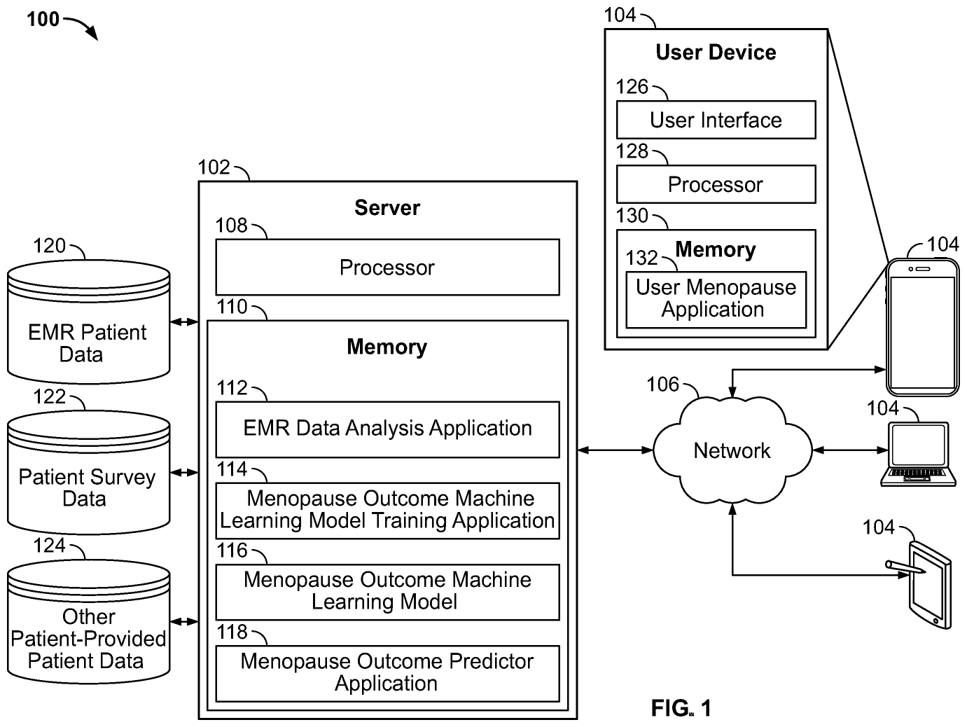

FIG. 1 illustrates a block diagram of an example system 100 for training a machine learning model to predict a menopause outcome trajectory for patients based on patient parameters, in accordance with some examples. The high-level architecture illustrated in FIG. 1 may include both hardware and software applications, as well as various data communications channels for communicating data between the various hardware and software components, as is described below.

The system 100 may include a server 102, and one or more user computing devices 104 (which may include, e.g., smart phones, tablets, personal computers, smart watches, etc.). The computing devices 102 and 104 may communicate with one another via a network 106, which may be a wired or wireless network. Generally speaking, the server 102 may include one or more processors 108 and a memory 110 (e.g., volatile memory, non-volatile memory) accessible by the one or more processors 108 (e.g., via a memory controller). The one or more processors 108 may interact with the memory 110 to obtain, for example, computer-readable instructions stored in the memory 110. The computer-readable instructions stored in the memory 110 may cause the one or more processors 108 to execute one or more applications, including, e.g., an electronic medical record (EMR) data analysis application 112, a menopause outcome machine learning model training application 114 that trains a menopause outcome machine learning model 116, and/or a menopause outcome predictor application 118.

For instance, executing the EMR data analysis application 112 may include analyzing EMR data associated with historical patients, e.g., as may be stored in an EMR patient database 120 populated by data from healthcare providers associated with the historical patients. For example, this analysis may include identifying codes in the EMR data that are associated with various patient parameters and/or menopause outcome trajectories associated with each historical patient, or analyzing notes written by healthcare providers in the EMR data using natural language processing (NLP) techniques to identify healthcare provider notes that are associated with patient parameters and/or menopause outcome trajectories associated with each historical patient.

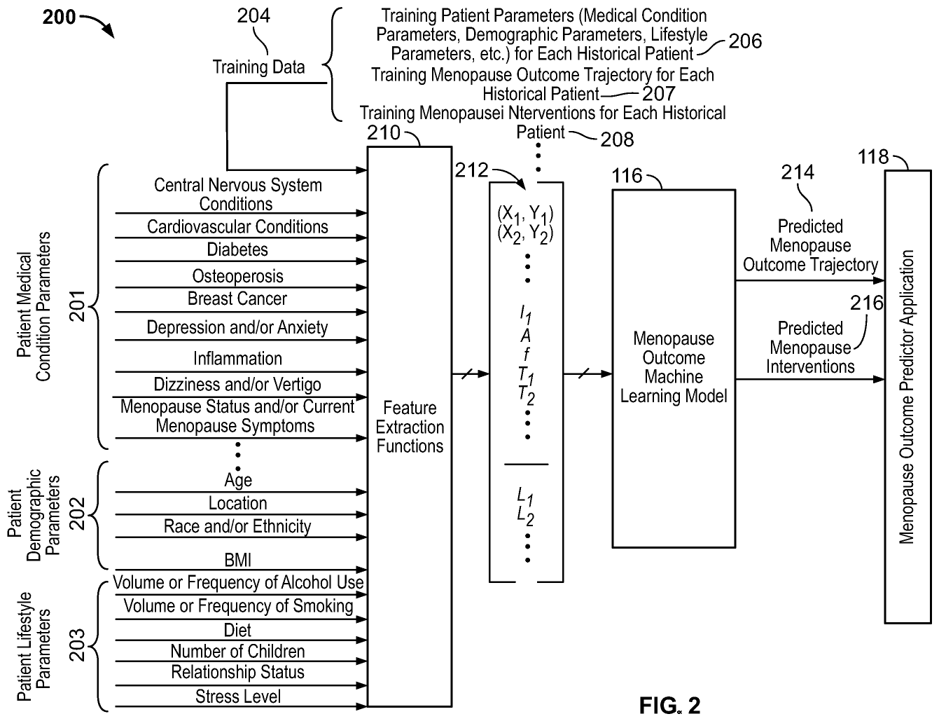

Executing the menopause outcome machine learning model training application 114 may include creating a training dataset including the patient parameters and menopause outcome trajectories associated with some number of historical patients (e.g., obtained via the analysis of EMR data by the EMR data analysis application 112, or obtained via patient surveys, as may be stored in the patient survey database 122, or otherwise provided by patients, as may be stored in the patient-provided patient database 124), and using the training dataset to train the menopause outcome machine learning model 116 to predict menopause outcome trajectories for patients based on patient parameters, as discussed in greater detail with respect to FIG. 2, below. In some examples, executing the menopause outcome machine learning model training application 114 may further include adding indications of treatments that were used to treat menopause symptoms of historical patients and indications of the success of each treatment in alleviating the symptoms of the historical patients, to the training dataset (i.e., in addition to the patient parameters and the patient menopause outcome trajectories associated with the historical patients), and using the training dataset to train the menopause outcome machine learning model 116 (e.g., in a similar manner as is shown with respect to FIG. 2) to further predict the likelihood of success of various treatments for alleviating symptoms indicated by patients' respective menopause outcome trajectories.

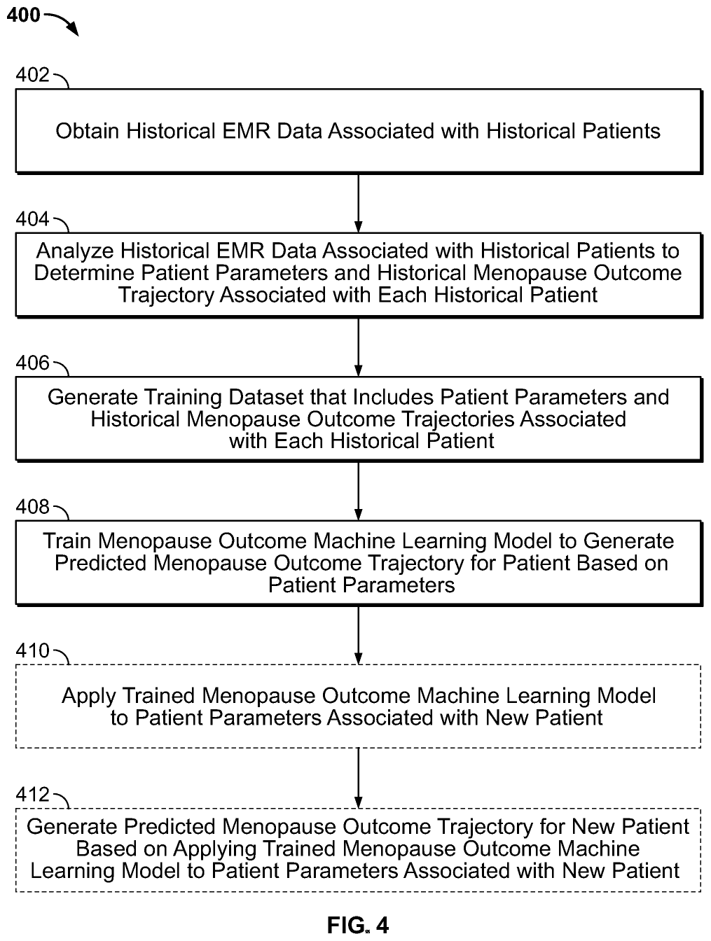

Additionally, executing the menopause outcome predictor application 118 may include applying the trained menopause outcome machine learning model 116 to patient parameters associated with a new patient in order to predict a menopause trajectory for that patient, and/or to predict a likelihood of success of various treatments for alleviating symptoms indicated by the menopause trajectory for that patient. Furthermore, in some examples, the computer-readable instructions stored on the memory 110 may include instructions for carrying out any of the steps of the method 400, described in greater detail below with respect to FIG. 4.

The user computing device(s) 104 may each include a user interface 126 which may be configured to receive inputs from users provide information to users (e.g., such as the predicted menopause outcome trajectory 214 discussed above), one or more processors 128, and a memory 130 (e.g., volatile memory, non-volatile memory) accessible by the one or more processors 128 (e.g., via a memory controller). The one or more processors 128 may interact with the memory 130 to obtain, for example, computer-readable instructions stored in the memory 130. The computer-readable instructions stored in the memory 130 may cause the one or more processors 128 to execute one or more applications, including a user menopause application 132.

For instance, executing the user menopause application 132 may include indications of various patient parameters from a patient, sending the patient parameters to the server 102, receiving a predicted menopause outcome trajectory for the patient and/or predicted likelihood of various treatments for alleviating symptoms indicated by the predicted menopause outcome trajectory for the patient (i.e., generated by the menopause outcome predictor application 118) from the server 102, and displaying the predicted menopause outcome trajectory (and/or predicted likelihood of the various treatments) for the patient via the user interface 126.

That is, in some examples, a patient who is not currently experiencing menopause or perimenopause may provide patient parameters as inputs to the user menopause application 132 via the user interface 126 and may receive a personalized predicted menopause outcome trajectory via the user interface 126. The personalized predicted menopause outcome trajectory may include one or more of a predicted age of onset of perimenopause, menopause, and/or post-menopause for the patient, particular menopause symptoms that the patient is likely to experience, an age of onset for each of the particular menopause symptoms, a severity level of each of the particular menopause symptoms, and/or other factors that characterize the historical patient's menopause experience over time. For example, for a patient who is black, a non-smoker, and 41 years old, who is currently experiencing headache and menstrual changes, the predicted menopause outcome trajectory may include a predicted age of onset of menopause at 45 years old, and predicted symptoms including continued (low severity level) menstrual changes, (medium severity level) mood changes, and a (low severity level) slowing metabolism. As another example, for a patient who is white, a smoker, and 48 years old, who is currently experiencing hot flashes and incontinence, the predicted menopause outcome trajectory may include a predicted age of onset of menopause at 49 years old, and predicted symptoms including continued (medium severity level) hot flashes, increasing (high severity level) incontinence, (low severity level) brain fog, and (low severity level) hair loss.

In some examples, executing the user menopause application 132 may further include providing indications of possible treatments for alleviating each symptom indicated by the predicted menopause outcome trajectory for the patient (i.e., in some instances indicating predicted likelihoods of success for each possible treatment), along with informational resources related to the symptoms (e.g., websites, books, videos, audio resources, etc.), and/or contact information for healthcare providers who may be able to treat the menopause symptoms or provide additional menopause information to the patient. Moreover, in some examples, executing the user menopause application 132 may include collecting data from patients, i.e., including indications of the ages at which a given patient experiences perimenopause, menopause, and/or post-menopause, ages at which the patient experiences various symptoms, the specific symptoms experienced by the patient and their severity, any treatments or interventions the patient has attempted to treat the various symptoms, indications of whether the treatments or interventions were successful, etc., and sending the collected data to the server 102 to be stored in the patient-provided patient database 124, i.e., to be used in training the machine learning model 116.

Once trained, the machine learning model 116 may take feedback on patient preferences and willingness to take actions from an application to improve suggestions and prioritize outcomes. Moreover, executing the user menopause application 132 may include analyzing patient inputs indicative of patient preference in order to provide indications of possible treatments for alleviating symptoms that combine psychology and behavioral economics to incentivize healthy choices. In some cases, the user menopause application 132 may retain users' historical preferences to inform future suggestions. For example, if a user is unwilling to take hormone replacement therapy (e.g., as indicated via user input), the user menopause application 132 may prioritize other solutions. Similarly, if a user prioritizes weight loss over mood (e.g., as indicated via user input), the user menopause application 132 may provide solutions that reflect that prioritization. The user menopause application 132 may use prioritization to provide incentives that impact not only those priorities but also the user's overall, long term health. Generally speaking, the user menopause application 132 may be prevented from suggesting poor health choices and instead may provide a recommendation sending the patient for a physician consultation to discuss the risks and benefits of various treatment methods.

Furthermore, in some examples, the computer-readable instructions stored on the memory 130 may include instructions for carrying out any of the steps of the method 400, described in greater detail below with respect to FIG. 4.

Now referring to FIG. 2, as discussed above, the menopause outcome machine learning model training application 114 may train the menopause outcome machine learning model 116 in accordance with the scheme 200, and the menopause predictor application 118 may operate the trained menopause outcome machine learning model 116 in accordance with the scheme 200. The menopause outcome machine learning model training application 114 can receive various input signals, including patient parameters for a new patient. For instance, the patient parameters may include one or more of patient medical condition parameters 201, patient demographic parameters 202, and/or patient lifestyle parameters 203 for a new patient (i.e., for whom a menopause trajectory is to be predicted), as well as training data 204 obtained via the analysis of EMR data by the EMR data analysis application 112, or obtained via patient surveys, as may be stored in the patient survey database 122, or otherwise provided by patients, as may be stored in the patient-provided patient database 124. For instance, the training data 204 may include training patient parameters 206 (i.e., including training patient medical condition parameters, patient demographic parameters, and/or patient lifestyle parameters) for a plurality of historical patients, and a historical menopause trajectory 207 associated with each of the historical patients. In some examples, the training data 204 may further include indications of historical interventions 208 or treatments used by historical patients to prevent and/or alleviate menopause symptoms, as well as indications of the effectiveness or successfulness of the historical interventions 208 or treatments.

The patient parameters and the training patient parameters 206 may each include patient medical condition parameters (e.g., 201) patient demographic parameters, (e.g., 202) and/or patient lifestyle parameters (e.g., 203). The patient medical condition parameters (e.g., 201 and/or 206) may include any previous or current medical conditions or symptoms associated with the patient, such as, e.g., number or frequency of hot flashes, sleep disruption, fatigue, sexual dysfunction, vaginal dryness, presence of menstruation, characteristics of menstruation, instances of amenorrhea, night sweats, urinary symptoms, mood symptoms, brain fog, changes in ability to focus, hair loss, weight gain, perimenopause or menopause status, arthritis, depression, anxiety, breast cancer, osteoporosis, dizziness, vertigo, inflammation, cardiovascular conditions, diabetes, central nervous system conditions, etc., experienced by the patient. In some examples, additional or alternative medical conditions or symptoms associated with the patient may be included as medical condition parameters 201 and/or 206. For instance, in some examples, the medical condition parameters 201 and/or 206 may include irregular periods, mood swings, bloating, sore breasts, changes in libido, headaches, pain in joints, electric shock sensations, burning tongue, gum problems, digestive issues, dry and itchy skin, tingling sensation in the extremities, sleeping difficulties, difficulty concentrating, fatigue, memory lapse, brittle nails, tight muscles, stress incontinence, changes in body odor, irritability, allergies, irregular heartbeat, panic disorder, and/or osteoporosis.

Patient demographic parameters (e.g., 202 and/or 206) may include e.g., the age associated with the patient (e.g., the patient's current age, or an age at which one of the previous medical conditions or symptoms was experienced by the patient), a location associated with the patient (e.g., the country, state, county, or city where the patient lives or where the patient's medical provider is located), and/or a race or ethnicity associated with the patient. Finally, patient lifestyle parameters (e.g., 203 and/or 206) may include the patient's body mass index or changes thereof, a volume or frequency of the patient's alcohol use, a duration or frequency of the patient's smoking use, the patient's diet or various changes thereto, the number of children the patient has, the patient's relationship status, and/or some measure of the patient's stress level.

The training menopause outcome trajectory 207 for each historical patient may include an age of onset of perimenopause for the historical patient, an age of onset of menopause for the historical patient, an age of onset of post-menopause for the historical patient, particular menopause symptoms experienced by the historical patient, an age of onset of each menopause symptom experienced by the historical patient, and/or a severity level associated with each menopause symptom experienced by the historical patient, and/or other factors that characterize the historical patient's menopause experience over time.

The training menopause interventions 208 for each historical patient may include indications of treatments or lifestyle changes attempted by the historical patient, and/or indications of their effectiveness in alleviating and/or preventing various menopause symptoms.

Generally speaking, the feature extraction functions 210 can operate on at least some of these input signals to generate feature vectors, or logical groupings of parameters associated with various patient parameters for each historical patient's menopause trajectory. For example, the feature extraction functions 210 may generate a feature vector that indicates that for a patient who has experienced cardiovascular conditions, the result corresponds to an earlier age onset of menopause. As another example, the feature extraction functions 210 may generate a feature vector that indicates that for a patient who has had diabetes and undergone diabetes treatment, the feature extraction functions 210 may generate a feature vector that indicates a later age of onset of menopause. These results can be used as a set of labels for the feature vectors.

Accordingly, the feature extraction functions 210 can generate feature vectors 212 using the training patient parameters 206 for each historical patient, the training menopause outcome trajectories 207, and the training menopause interventions 208 for each historical patient. In general, the menopause outcome machine learning model training application 114 can train the menopause outcome machine learning model 116 using supervised learning, unsupervised learning, reinforcement learning, or any other suitable technique. Moreover, the menopause outcome machine learning model training application 114 can train the menopause outcome machine learning model 116 as a standard regression model.

Over time, as the menopause outcome machine learning model training application 114 can train the menopause outcome machine learning model 116, the trained menopause outcome machine learning model 116 may learn to predict a menopause outcome trajectory 214 and/or learn to predict the effectiveness of one or more menopause interventions 216 for a given patient based on patient parameters 201, 202, and/or 203 associated with the patient. For instance, the menopause outcome predictor application 118 may receive patient parameters 201, 202, and/or 203 for a new patient as inputs (e.g., via a user interface of the user computing device 104), and may apply the trained menopause outcome machine learning model 116 to the patient parameters 201, 202, and/or 203 for the new patient. The trained menopause outcome machine learning model 116 may then generate a predicted menopause outcome trajectory 214 and/or a predicted effectiveness of one or more menopause interventions 216 for the new patient using the patient parameters 201, 202 and/or 203, and may send an indication of the predicted menopause outcome trajectory 214 and/or the predicted effectiveness of one or more menopause interventions 216 to the menopause outcome predictor application 118, which may display the predicted menopause outcome trajectory 214 and/or the predicted effectiveness of one or more menopause interventions 216 to a user, or may send the predicted menopause outcome trajectory 214 the predicted effectiveness of one or more menopause interventions 216 to another device (such as the user computing device 104) to be displayed to a user.

As the user menopause application 130 collects new patient data from patients and stores this data in the patient-provided patient database 124, this data may be used in subsequent training of the menopause outcome machine learning model 116, i.e., for fine-tuning to improve the performance of the menopause outcome machine learning model 116. For instance, the new patient data collected from patients may be analyzed to determine the accuracy of the predictions of the menopause outcome machine learning model 116, i.e., to determine whether a given patient's menopause outcome trajectory actually includes the patient experiencing menopause at the age predicted by the menopause outcome machine learning model 116, or experiencing the symptoms predicted by the menopause outcome machine learning model 116. As another example, the new patient data collected from patients may be analyzed to determine the accuracy of the predicted effectiveness the one or more menopause interventions 216 in preventing or alleviating various menopause symptoms experienced by the patient. This accuracy data, along with indications of actual versions of the predicted values (e.g., the actual age at which a given patient experiences menopause, compared to the age predicted by the model; the actual interventions that are most effective at preventing or alleviating the symptoms experienced by the patient compared to the predicted interventions or the predicted effectiveness of those interventions; etc.) may then be used as further training data 204 for training the machine learning model 116.

In some examples, patient outcomes (e.g., paths or trajectories) may be determined by a highly dimensional time series clustering and trajectory clustering ensemble. The methods are trained on a combined EMR and claims dataset with two holdouts. The first holdout is used to judge and tune the individual segmentation models. The second is used to set the weights across the models or select the best segmentation. The segmented paths may be coded into Bayesian Networks to assess how treatment decisions and behavioral changes could improve patients' quality of life or slow progression on their current path, or shift them to a new, healthier path. Reinforcement learning will be used with new datasets and physician feedback through a survey application to update the Bayesian priors as evidence builds.

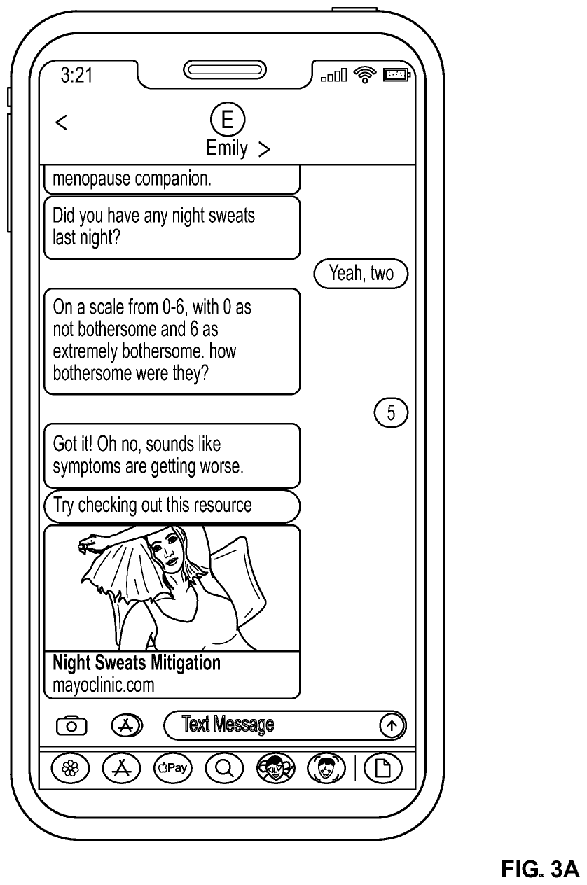

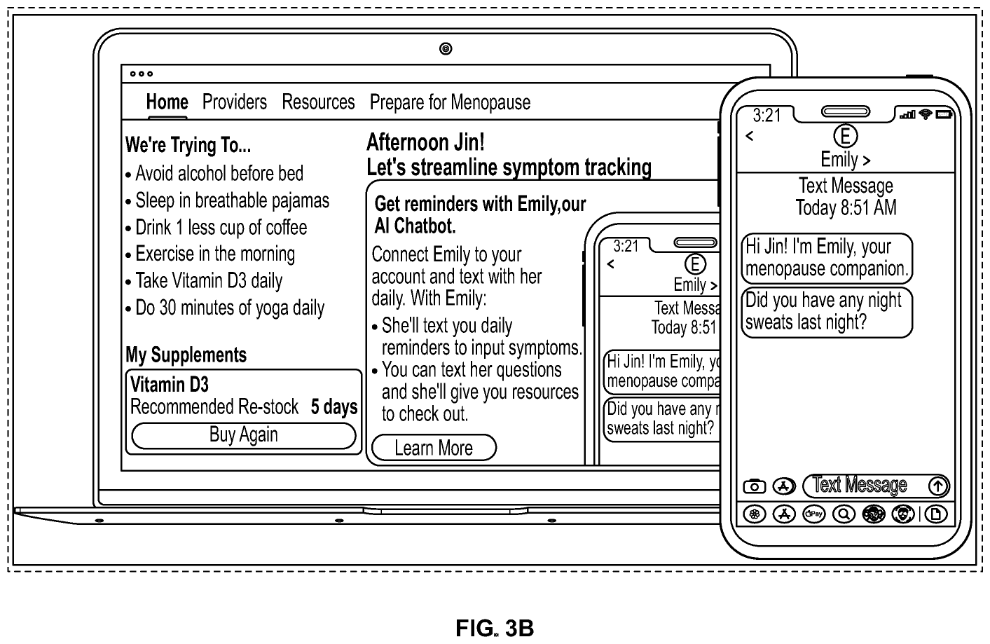

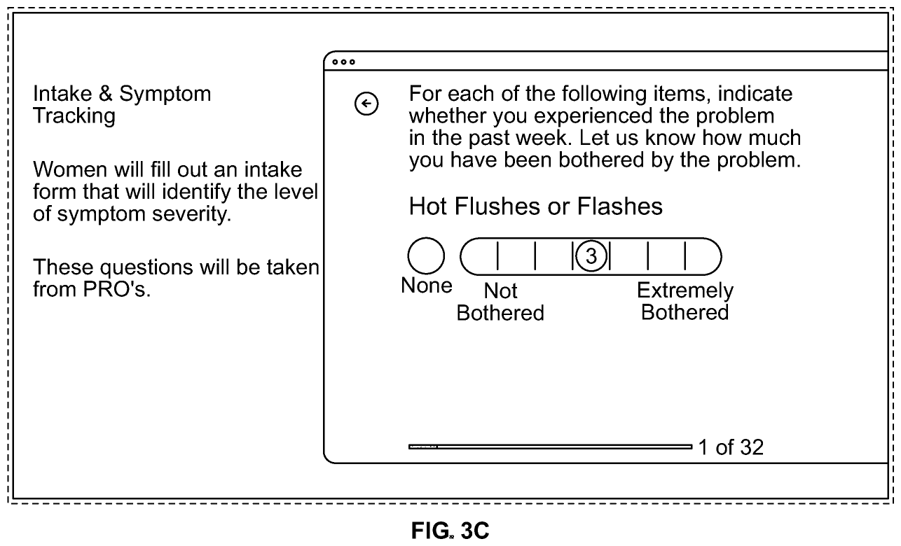

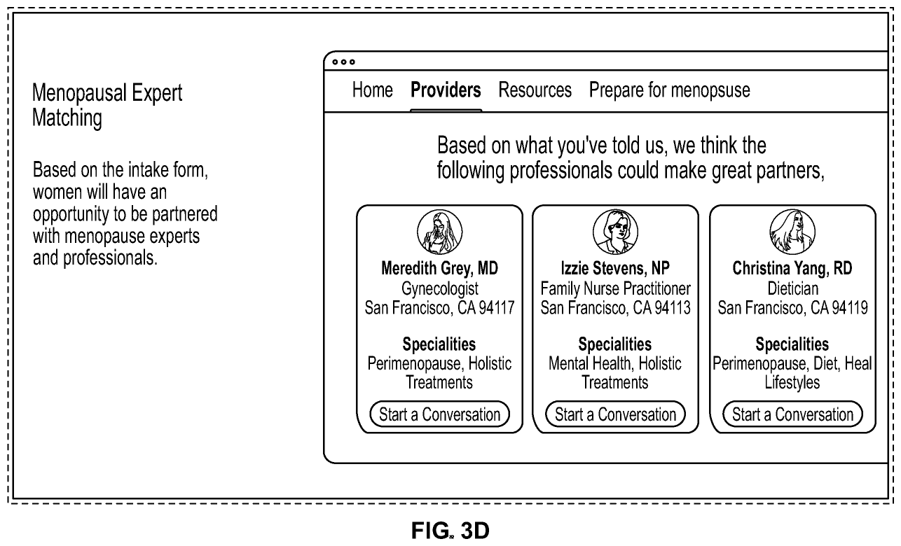

FIGS. 3A-3E illustrate several example user interface displays for the user menopause application 132, as may be displayed via the user interface 126, in accordance with some examples. For instance, FIG. 3A illustrates an example user interface display that may be used by the user menopause application 132 to provide resources for a particular symptom, such as an article about night sweats, to a patient. FIG. 3B illustrates an example user interface display that may be used by the user menopause application 132 to request patient parameters (e.g., patient medical parameters including current symptoms) from a patient and provide a listing of possible treatments for the current symptoms. Similarly, FIG. 3C illustrates an example user interface display that may be used by the user menopause application 132 to receive an indication of the severity of one or more current symptoms experienced by patients. Finally, FIG. 3D illustrates an example user interface display that may be used by the user menopause application 132 to provide contact information for healthcare providers who may be able to treat the menopause symptoms or provide additional menopause information to the patient.

FIG. 4 illustrates a flow diagram of an example method 400 for training a machine learning model to predict a menopause outcome trajectory for patients based on patient parameters, in accordance with some examples. One or more steps of the method 400 may be implemented as a set of instructions stored on a computer-readable memory (e.g., memories 110 or 130) and executable on one or more processors (e.g., processors 108 or 128).

The method 400 may begin when historical electronic medical record (EMR) data associated with a plurality of historical patients is obtained (block 402). The historical EMR data associated with the plurality of historical patients may be analyzed (block 404) to determine one or more patient parameters associated with each historical patient and a historical menopause outcome trajectory associated with each historical patient. For instance, analyzing the historical EMR data associated with the plurality of historical patients may include identifying codes in the EMR data that are associated with patient parameters and/or historical menopause outcome trajectories associated with each historical patient. Moreover, in some examples, analyzing the historical EMR data associated with the plurality of historical patients may include analyzing notes written by healthcare providers in the EMR data using natural language processing (NLP) techniques to identify healthcare provider notes that are associated with patient parameters and/or menopause outcome trajectories associated with each historical patient.

The patient parameters may include, e.g., patient medical condition parameters, patient demographic parameters, and/or patient lifestyle parameters. The patient medical condition parameters include previous or current medical conditions or symptoms experienced by or otherwise associated with the patient. E.g., the number or frequency of hot flashes, sleep disruption, fatigue, sexual dysfunction, vaginal dryness, presence of menstruation, characteristics of menstruation, instances of amenorrhea, night sweats, urinary symptoms, mood symptoms, brain fog, changes in ability to focus, hair loss, weight gain, perimenopause or menopause status, arthritis, depression, anxiety, breast cancer, osteoporosis, dizziness, vertigo, inflammation, cardiovascular conditions, diabetes, nervous system conditions, etc. In some examples, additional or alternative medical conditions or symptoms associated with the patient may be included as medical condition parameters. For instance, in some examples, the medical condition parameters may include irregular periods, mood swings, bloating, sore breasts, changes in libido, headaches, pain in joints, electric shock sensations, burning tongue, gum problems, digestive issues, dry and itchy skin, tingling sensation in the extremities, sleeping difficulties, difficulty concentrating, fatigue, memory lapse, brittle nails, tight muscles, stress incontinence, changes in body odor, irritability, allergies, irregular heartbeat, panic disorder, and/or osteoporosis.

The patient demographic parameters may include, e.g., the age associated with the patient (e.g., the patient's current age, or an age at which one of the previous medical conditions or symptoms was experienced by the patient), a location associated with the patient (e.g., the country, state, county, or city where the patient lives or where the patient's medical provider is located), and/or a race or ethnicity associated with the patient.

The patient lifestyle parameters may include, e.g., the patient's body mass index or changes thereof, a volume or frequency of the patient's alcohol use, a duration or frequency of the patient's smoking use, the patient's diet or various changes thereto, the number of children the patient has, the patient's relationship status, or some measure of the patient's stress level.

The patient's menopause outcome trajectory may include an age of onset of perimenopause for the patient, an age of onset of menopause for the patient, an age of onset of post-menopause for the patient, particular menopause symptoms experienced by the patient, an age of onset of each menopause symptom experienced by the patient, and/or a severity level associated with each menopause symptom experienced by the patient, or other factors that characterize the patient's menopause experience over time.

A training dataset may generated (block 406) using the patient parameters and menopause outcome trajectories associated with each historical patient from the EMR. In some examples, the training dataset may be generated using additional or alternative sources of patient parameters and menopause outcome trajectories associated with historical patients. For instance, these sources may include survey data from historical patients, or data received from historical patients, e.g., via a user mobile device application.

Using the training dataset, a menopause outcome machine learning model may be trained (block 408), to generate a predicted menopause outcome trajectory for patients based on patient parameters. That is, as discussed above, the predicted menopause outcome trajectory may include a predicted age of onset of perimenopause for the patient, a predicted age of onset of menopause for the patient, a predicted age of onset of post-menopause for the patient, particular menopause symptoms predicted to be experienced by the patient, an age of onset of each menopause symptom predicted to be experienced by the patient, and/or a severity level associated with each menopause symptom predicted to be experienced by the patient, or other factors that characterize the patient's menopause experience over time. For instance, the menopause symptoms predicted to be experienced by the patient may include the number or frequency of hot flashes, sleep disruption, fatigue, sexual dysfunction, vaginal dryness, presence of menstruation, characteristics of menstruation, instances of amenorrhea, night sweats, urinary symptoms, mood symptoms, brain fog, changes in ability to focus, hair loss, weight gain, perimenopause or menopause status, arthritis, depression, anxiety, breast cancer, osteoporosis, dizziness, vertigo, inflammation, cardiovascular conditions, diabetes, nervous system conditions, etc. In some examples, the predicted menopause outcome trajectory may include additional or alternative symptoms predicted to be experienced by the patient. For instance, in some examples, these symptoms may include irregular periods, mood swings, bloating, sore breasts, changes in libido, headaches, pain in joints, electric shock sensations, burning tongue, gum problems, digestive issues, dry and itchy skin, tingling sensation in the extremities, sleeping difficulties, difficulty concentrating, fatigue, memory lapse, brittle nails, tight muscles, stress incontinence, changes in body odor, irritability, allergies, irregular heartbeat, panic disorder, and/or osteoporosis.

In some examples, the method 400 may further include applying (block 410) the trained menopause outcome machine learning model to one or more patient parameters associated with a new patient; and predicting (block 412) a menopause outcome trajectory associated with the new patient based on applying the trained menopause outcome machine learning model to the one or more patient parameters associated with the new patient.

Furthermore, in some examples, the method 400 may include analyzing the historical EMR data associated with the plurality of historical patients to determine one or more menopause symptom treatments associated with each historical patient and including this information in the generation of the training dataset when training wherein training the menopause outcome machine learning model. Accordingly, using the training dataset, the menopause outcome machine learning model may also be trained to predict menopause symptom treatments for alleviating the predicted menopause outcome trajectory. That is, in some examples, the trained menopause outcome machine learning model may also be applied to the one or more patient parameters associated with the new patient to predict menopause symptom treatments that may alleviate or prevent one or more symptoms of the predicted menopause outcome trajectory for the patient.

Examples of some menopause symptom treatments that may alleviate or otherwise change the predicted menopause outcome trajectory for the patient include: biologics, steroids, nonsteroidal anti-inflammatory drugs (NSAIDs), gabapentin or pregablin, leuperelin, hormone therapy, chemotherapy, metformin, sodium-glucose co-transporter-2 (SGLT2), peroxisome proliferator-activated receptors (PPAR), sulfonylureas, dipeptidyl-peptidase 4 (DPP4), insulin, orlistat, synthetic thyroid, statins, glucagon-like peptide-1 (GLP-1), angiotensin II receptor blockers (ARBs), calcium channel blockers (CCBs), angiotensin converting enzyme (ACE), diuretics, ambien, selective serotonin reuptake inhibitors (SSRIs), norepinephrine and dopamine reuptake inhibitors (NDRI), monoamine oxidase inhibitors (MAOIs), oral contraceptives, hormone replacement therapy, topical or vaginal hormone creams, natural or herbal remedies, cognitive therapy, exercise, meditation, vaginal lubricants, diet alterations, psychotherapy, vitamin D, and/or clonidine.

### Aspects

Embodiments of the techniques described in the present disclosure may include any number of the following aspects, either alone or combination:

1. A computer-implemented method, comprising: obtaining, by one or more processors, historical electronic medical record (EMR) data associated with a plurality of historical patients; analyzing, by the one or more processors, the historical EMR data associated with the plurality of historical patients to determine one or more patient parameters associated with each historical patient and a historical menopause outcome trajectory associated with each historical patient; generating, by the one or more processors, a training dataset that includes patient parameters and historical menopause outcome trajectories associated with each historical patient; and training, by the one or more processors, a menopause outcome machine learning model, using the training dataset, to generate a predicted menopause outcome trajectory for a patient based on patient parameters associated with the patient, wherein the predicted menopause outcome trajectory includes or consists of predictions of one or more of: an age of onset of perimenopause for the patient, an age of onset of menopause for the patient, an age of onset of post-menopause for the patient, particular menopause symptoms experienced by the patient, an age of onset of each menopause symptom experienced by the patient, or a severity level associated with each menopause symptom experienced by the patient.

2. The computer-implemented method of aspect 1, further comprising: applying, by the one or more processors, the trained menopause outcome machine learning model to one or more patient parameters associated with a new patient; and generating, by the one or more processors, based on applying the trained menopause outcome machine learning model to the one or more patient parameters associated with the new patient, a predicted menopause outcome trajectory associated with the new patient.

3. The computer-implemented method of any one of aspects 1 or 2, wherein analyzing the historical EMR data associated with the plurality of historical patients to determine one or more patient parameters associated with each historical patient and a historical menopause outcome trajectory associated with each historical patient includes analyzing the historical EMR data using natural language processing (NLP) techniques.

4. The computer-implemented method of any one of aspects 1-3, wherein the patient parameters include one or more of: patient medical condition parameters, patient demographic parameters, or patient lifestyle parameters.

5. The computer-implemented method of aspect 4, wherein the patient medical condition parameters include previous or current medical conditions or symptoms associated with the patient, including one or more of: number or frequency of hot flashes, sleep disruption, fatigue, sexual dysfunction, vaginal dryness, presence of menstruation, characteristics of menstruation, instances of amenorrhea, night sweats, urinary symptoms, mood symptoms, brain fog, changes in ability to focus, hair loss, weight gain, perimenopause or menopause status, arthritis, depression, anxiety, breast cancer, osteoporosis, dizziness, vertigo, inflammation, cardiovascular conditions, diabetes, or central nervous system conditions experienced by the patient.

6. The computer-implemented method of any one of aspects 4 or 5, wherein the patient demographic parameters include one or more of: an age, a location, or a race or ethnicity associated with the patient.

7. The computer-implemented method of any one of aspects 4-6, wherein the patient lifestyle parameters include one or more of: a body mass index, a volume or frequency of alcohol use, a duration or frequency of smoking use, a diet, a number of children, a relationship status, or a stress level associated with the patient.

The computer-implemented method of any one of aspects 1-7, wherein the menopause symptoms of the historical menopause outcome trajectory and the predicted menopause trajectory each include one or more of: number or frequency of hot flashes, sleep disruption, fatigue, sexual dysfunction, vaginal dryness, presence of menstruation, characteristics of menstruation, instances of amenorrhea, night sweats, urinary symptoms, mood symptoms, brain fog, changes in ability to focus, hair loss, weight gain, depression, anxiety, dizziness, vertigo, inflammation, or central nervous system conditions experienced by the patient.

9. The computer-implemented method of any one of aspects 1-8, further comprising analyzing the historical EMR data associated with the plurality of historical patients to determine one or more menopause symptom treatments associated with each historical patient; wherein the training dataset includes menopause symptom treatments associated with each historical patient, and wherein training the menopause outcome machine learning model to generate the predicted menopause outcome trajectory for a patient based on patient parameters associated with the patient includes training the menopause outcome machine learning model to generate predicted menopause symptom treatments for alleviating or preventing one or more symptoms of the predicted menopause outcome trajectory.

10. The computer-implemented method of aspect 9, wherein the menopause symptom treatments include one or more of: biologics, steroids, nonsteroidal anti-inflammatory drugs (NSAIDs), gabapentin or pregablin, leuperelin, hormone therapy, chemotherapy, metformin, sodium-glucose co-transporter-2 (SGLT2), peroxisome proliferator-activated receptors (PPAR), sulfonylureas, dipeptidyl-peptidase 4 (DPP4), insulin, orlistat, synthetic thyroid, statins, glucagon-like peptide-1 (GLP-1), angiotensin II receptor blockers (ARBs), calcium channel blockers (CCBs), angiotensin converting enzyme (ACE), diuretics, ambien, selective serotonin reuptake inhibitors (SSRIs), norepinephrine and dopamine reuptake inhibitors (NDRI), monoamine oxidase inhibitors (MAOIs), oral contraceptives, hormone replacement therapy, topical or vaginal hormone creams, natural or herbal remedies, cognitive therapy, exercise, meditation, vaginal lubricants, diet alterations, psychotherapy, vitamin D, or clonidine.

11. A computer system, comprising: one or more processors; and a non-transitory program memory communicatively coupled to the one or more processors and storing executable instructions that, when executed by the one or more processors, cause the processors to: obtain historical electronic medical record (EMR) data associated with a plurality of historical patients; analyze the historical EMR data associated with the plurality of historical patients to determine one or more patient parameters associated with each historical patient and a historical menopause outcome trajectory associated with each historical patient; generate a training dataset that includes patient parameters and menopause outcome trajectories associated with each historical patient; and train a menopause outcome machine learning model, using the training dataset, to generate a predicted menopause outcome trajectory for patients based on patient parameters associated with the patient, wherein the predicted menopause outcome trajectory includes or consists of predictions of one or more of: an age of onset of perimenopause for the patient, an age of onset of menopause for the patient, an age of onset of post-menopause for the patient, particular menopause symptoms experienced by the patient, an age of onset of each menopause symptom experienced by the patient, or a severity level associated with each menopause symptom experienced by the patient.

12. The computer system of aspect 11, wherein the executable instructions, when executed by the one or more processors, further cause the processors to: apply the trained menopause outcome machine learning model to one or more patient parameters associated with a new patient; and generate, based on applying the trained menopause outcome machine learning model to the one or more patient parameters associated with the new patient, a predicted menopause outcome trajectory associated with the new patient.

13. The computer system of any one of aspects 11 or 12, wherein the executable instructions, when executed by the one or more processors, cause the processors to analyze the historical EMR data associated with the plurality of historical patients to determine one or more patient parameters associated with each historical patient and a historical menopause outcome trajectory associated with each historical patient using natural language processing (NLP) techniques.

14. The computer system of any one of aspects 11-13, wherein the patient parameters include one or more of: patient medical condition parameters, patient demographic parameters, or patient lifestyle parameters.

15. The computer system of aspect 14, wherein the patient medical condition parameters include previous or current medical conditions or symptoms associated with the patient, including one or more of: number or frequency of hot flashes, sleep disruption, fatigue, sexual dysfunction, vaginal dryness, presence of menstruation, characteristics of menstruation, instances of amenorrhea, night sweats, urinary symptoms, mood symptoms, brain fog, changes in ability to focus, hair loss, weight gain, perimenopause or menopause status, arthritis, depression, anxiety, breast cancer, osteoporosis, dizziness, vertigo, inflammation, cardiovascular conditions, diabetes, or central nervous system conditions experienced by the patient.

16. The computer system of any one of aspects 14 or 15, wherein the wherein the patient demographic parameters include one or more of: an age, a location, or a race or ethnicity associated with the patient.

17. The computer system of any one of aspects 14-16, wherein the patient lifestyle parameters include one or more of: a body mass index, a volume or frequency of alcohol use, a duration or frequency of smoking use, a diet, a number of children, a relationship status, or a stress level associated with the patient.

18. The computer system of any one of aspects 11-17, wherein the menopause symptoms of the historical menopause outcome trajectory and the predicted menopause outcome trajectory each include one or more of: number or frequency of hot flashes, sleep disruption, fatigue, sexual dysfunction, vaginal dryness, presence of menstruation, characteristics of menstruation, instances of amenorrhea, night sweats, urinary symptoms, mood symptoms, brain fog, changes in ability to focus, hair loss, weight gain, depression, anxiety, dizziness, vertigo, inflammation, or central nervous system conditions experienced by the patient.

19. The computer system of any one of aspects 11-18, wherein the executable instructions, when executed by the one or more processors, further cause the processors to analyze the historical EMR data associated with the plurality of historical patients to determine one or more menopause symptom treatments associated with each historical patient; and wherein the training dataset includes menopause symptom treatments associated with each historical patient, and wherein training the menopause outcome machine learning model to generate the predicted menopause outcome trajectory for a patient based on patient parameters associated with the patient includes training the menopause outcome machine learning model to predict menopause symptom treatments for alleviating or preventing one or more menopause symptoms of the predicted menopause outcome trajectory.

20. The computer system of aspect 19, wherein the menopause symptom treatments include one or more of: biologics, steroids, nonsteroidal anti-inflammatory drugs (NSAIDs), gabapentin or pregablin, leuperelin, hormone therapy, chemotherapy, metformin, sodium-glucose co-transporter-2 (SGLT2), peroxisome proliferator-activated receptors (PPAR), sulfonylureas, dipeptidyl-peptidase 4 (DPP4), insulin, orlistat, synthetic thyroid, statins, glucagon-like peptide-1 (GLP-1), angiotensin II receptor blockers (ARBs), calcium channel blockers (CCBs), angiotensin converting enzyme (ACE), diuretics, ambien, selective serotonin reuptake inhibitors (SSRIs), norepinephrine and dopamine reuptake inhibitors (NDRI), monoamine oxidase inhibitors (MAOIs), oral contraceptives, hormone replacement therapy, topical or vaginal hormone creams, natural or herbal remedies, cognitive therapy, exercise, meditation, vaginal

21. A non-transitory computer readable storage medium storing computer-readable instructions that, when executed by one or more processors, cause the one or more processors to: obtain historical electronic medical record (EMR) data associated with a plurality of historical patients; analyze the historical EMR data associated with the plurality of historical patients to determine one or more patient parameters associated with each historical patient and a historical menopause outcome trajectory associated with each historical patient; generate a training dataset that includes patient parameters and menopause outcome trajectories associated with each historical patient; and train a menopause outcome machine learning model, using the training dataset, to generate a predicted menopause outcome trajectory for a patient based on patient parameters associated with the patient, wherein the predicted menopause outcome trajectory includes or consists of one or more of: an age of onset of perimenopause for the patient, an age of onset of menopause for the patient, an age of onset of post-menopause for the patient, particular menopause symptoms experienced by the patient, an age of onset of each menopause symptom experienced by the patient, or a severity level associated with each menopause symptom experienced by the patient.

22. The non-transitory computer readable storage medium of aspect 21, wherein the computer-readable instructions, when executed by the one or more processors, further cause the processors to: apply the trained menopause outcome machine learning model to one or more patient parameters associated with a new patient; and generate, based on applying the trained menopause outcome machine learning model to the one or more patient parameters associated with the new patient, a predicted menopause outcome trajectory associated with the new patient.

23. The non-transitory computer readable storage medium of any one of aspects 21 or 22, wherein the computer-readable instructions, when executed by the one or more processors, cause the processors to: analyze the historical EMR data associated with the plurality of historical patients to determine one or more patient parameters associated with each historical patient and a historical menopause outcome trajectory associated with each historical patient using natural language processing (NLP) techniques.

24. The non-transitory computer readable storage medium of any one of aspects 21-23, wherein the patient parameters include one or more of: patient medical condition parameters, patient demographic parameters, or patient lifestyle parameters.

25. The non-transitory computer readable storage medium of aspect 24, wherein the patient medical condition parameters include previous or current medical conditions or symptoms associated with the patient, including one or more of: number or frequency of hot flashes, sleep disruption, fatigue, sexual dysfunction, vaginal dryness, presence of menstruation, characteristics of menstruation, instances of amenorrhea, night sweats, urinary symptoms, mood symptoms, brain fog, changes in ability to focus, hair loss, weight gain, perimenopause or menopause status, arthritis, depression, anxiety, breast cancer, osteoporosis, dizziness, vertigo, inflammation, cardiovascular conditions, diabetes, or central nervous system conditions experienced by the patient.

26. The non-transitory computer readable storage medium of any one of aspects 24 or 25, wherein the patient demographic parameters include one or more of: an age, a location, or a race or ethnicity associated with the patient.

27. The non-transitory computer readable storage medium of any one of aspects 24-26, wherein the patient lifestyle parameters include one or more of: a body mass index, a volume or frequency of alcohol use, a duration or frequency of smoking use, a diet, a number of children, a relationship status, or a stress level associated with the patient.

28. The non-transitory computer readable storage medium of any one of aspects 24-27, wherein the menopause symptoms of the historical menopause outcome trajectory and the predicted menopause outcome trajectory each include one or more of: number or frequency of hot flashes, sleep disruption, fatigue, sexual dysfunction, vaginal dryness, presence of menstruation, characteristics of menstruation, instances of amenorrhea, night sweats, urinary symptoms, mood symptoms, brain fog, changes in ability to focus, hair loss, weight gain, depression, anxiety, dizziness, vertigo, inflammation, or central nervous system conditions experienced by the patient.

29. The non-transitory computer readable storage medium of any one of aspects 21-28, wherein the computer-readable instructions, when executed by the one or more processors, further cause the processors to analyze the historical EMR data associated with the plurality of historical patients to determine one or more menopause symptom treatments associated with each historical patient; and wherein the training dataset includes menopause symptom treatments associated with each historical patient, and wherein training the menopause outcome machine learning model to generate the predicted menopause outcome trajectory for a patient based on patient parameters includes training the menopause outcome machine learning model to predict menopause symptom treatments for preventing or alleviating one or more menopause symptoms of the predicted menopause outcome trajectory.

30. The non-transitory computer readable storage medium of aspect 29, wherein the menopause symptom treatments include one or more of: biologics, steroids, nonsteroidal anti-inflammatory drugs (NSAIDs), gabapentin or pregablin, leuperelin, hormone therapy, chemotherapy, metformin, sodium-glucose co-transporter-2 (SGLT2), peroxisome proliferator-activated receptors (PPAR), sulfonylureas, dipeptidyl-peptidase 4 (DPP4), insulin, orlistat, synthetic thyroid, statins, glucagon-like peptide-1 (GLP-1), angiotensin II receptor blockers (ARBs), calcium channel blockers (CCBs), angiotensin converting enzyme (ACE), diuretics, ambien, selective serotonin reuptake inhibitors (SSRIs), norepinephrine and dopamine reuptake inhibitors (NDRI), monoamine oxidase inhibitors (MAOIs), oral contraceptives, hormone replacement therapy, topical or vaginal hormone creams, natural or herbal remedies, cognitive therapy, exercise, meditation, vaginal lubricants, diet alterations, psychotherapy, vitamin D, or clonidine.

31. A computer-implemented method, comprising: obtaining, by one or more processors, patient parameters associated with a patient; analyzing, by the one or more processors, the patient parameters associated with the patient using a trained menopause outcome machine learning model; and generating, by the one or more processors, using the trained menopause outcome machine learning model, a predicted menopause outcome trajectory for the patient based on the patient parameters associated with the patient, wherein the menopause outcome trajectory includes or consists of one or more of: an age of onset of perimenopause for the patient, an age of onset of menopause for the patient, an age of onset of post-menopause for the patient, particular menopause symptoms experienced by the patient, an age of onset of each menopause symptom experienced by the patient, or a severity level associated with each menopause symptom experienced by the patient; and wherein the menopause outcome machine learning model is trained by: obtaining historical electronic medical record (EMR) data associated with a plurality of historical patients; analyzing the historical EMR data associated with the plurality of historical patients to determine one or more historical patient parameters associated with each historical patient and a historical menopause outcome trajectory associated with each historical patient; generating a training dataset that includes historical patient parameters and historical menopause outcome trajectories associated with each historical patient; and training the menopause outcome machine learning model, using the training dataset, to generate a predicted menopause outcome trajectory for new patients based on new patient parameters.

32. A computer system, comprising: one or more processors; and a non-transitory program memory communicatively coupled to the one or more processors and storing executable instructions that, when executed by the one or more processors, cause the processors to: obtain patient parameters associated with a patient; analyze the patient parameters associated with the patient using a trained menopause outcome machine learning model; and generate, using the trained menopause outcome machine learning model, a predicted menopause outcome trajectory for the patient based on the patient parameters associated with the patient, wherein the menopause outcome trajectory includes or consists of one or more of: an age of onset of perimenopause for the patient, an age of onset of menopause for the patient, an age of onset of post-menopause for the patient, particular menopause symptoms experienced by the patient, an age of onset of each menopause symptom experienced by the patient, or a severity level associated with each menopause symptom experienced by the patient; and wherein the menopause outcome machine learning model is trained by: obtaining historical electronic medical record (EMR) data associated with a plurality of historical patients; analyzing the historical EMR data associated with the plurality of historical patients to determine one or more historical patient parameters associated with each historical patient and a historical menopause outcome trajectory associated with each historical patient; generating a training dataset that includes historical patient parameters and historical menopause outcome trajectories associated with each historical patient; and training the menopause outcome machine learning model, using the training dataset, to generate a predicted menopause outcome trajectory for new patients based on new patient parameters.

33. A non-transitory computer readable storage medium storing computer-readable instructions that, when executed by one or more processors, cause the one or more processors to: obtain patient parameters associated with a patient; analyze the patient parameters associated with the patient using a trained menopause outcome machine learning model; and generate, using the trained menopause outcome machine learning model, a predicted menopause outcome trajectory for the patient based on the patient parameters associated with the patient, wherein the menopause outcome trajectory includes or consists of one or more of: an age of onset of perimenopause for the patient, an age of onset of menopause for the patient, an age of onset of post-menopause for the patient, particular menopause symptoms experienced by the patient, an age of onset of each menopause symptom experienced by the patient, or a severity level associated with each menopause symptom experienced by the patient; and wherein the menopause outcome machine learning model is trained by: obtaining historical electronic medical record (EMR) data associated with a plurality of historical patients; analyzing the historical EMR data associated with the plurality of historical patients to determine one or more historical patient parameters associated with each historical patient and a historical menopause outcome trajectory associated with each historical patient; generating a training dataset that includes historical patient parameters and historical menopause outcome trajectories associated with each historical patient; and training the menopause outcome machine learning model, using the training dataset, to generate a predicted menopause outcome trajectory for new patients based on new patient parameters.

In any one of aspects 1-33, the predicted menopause outcome trajectory can include or consist of predictions of: (a) one or more of (i) an age of onset of perimenopause for the patient, (ii) an age of onset of menopause for the patient, and (iii) an age of onset of post-menopause; and/or (b) one or more, two or more, 3 or more, 4 or more, 5 or more of particular menopause symptoms expected be experienced by the patient, such as a number or frequency of hot flashes, sleep disruption, fatigue, sexual dysfunction, vaginal dryness, presence of menstruation, characteristics of menstruation, instances of amenorrhea, night sweats, urinary symptoms, mood symptoms, brain fog, changes in ability to focus, hair loss, weight gain, depression, anxiety, dizziness, vertigo, inflammation, and/or central nervous system conditions, possibly together with the severity level associated with some or each/all of these symptoms.

In any one of aspects 1-33, the historical EMR data can comprise outcomes of laboratory tests (including hormone laboratory tests) prescribed to patients by health care professionals including one or more of: estradiol laboratory test, uric acid laboratory test, testosterone laboratory test, Hba1c laboratory test, presence or absence of abnormal hormone level tests, FSH laboratory test, or estrogen level laboratory test.

What is claimed is:

1. A computer-implemented method, comprising:
obtaining, by one or more processors, historical electronic medical record (EMR) data associated with a plurality of historical patients;
analyzing, by the one or more processors, the historical EMR data associated with the plurality of historical patients to determine one or more patient parameters associated with each historical patient and a historical menopause outcome trajectory associated with each historical patient;
generating, by the one or more processors, a training dataset that includes patient parameters and historical menopause outcome trajectories associated with each historical patient; and
training, by the one or more processors, a menopause outcome machine learning model, using the training dataset, to generate a predicted menopause outcome trajectory for a patient based on patient parameters associated with the patient, wherein the predicted menopause outcome trajectory includes or consists of predictions of one or more of: an age of onset of perimenopause for the patient, an age of onset of menopause for the patient, an age of onset of post-menopause for the patient, particular menopause symptoms experienced by the patient, an age of onset of each menopause symptom experienced by the patient, or a severity level associated with each menopause symptom experienced by the patient.

2. The computer-implemented method of claim 1, further comprising:
applying, by the one or more processors, the trained menopause outcome machine learning model to one or more patient parameters associated with a new patient; and
generating, by the one or more processors, based on applying the trained menopause outcome machine learning model to the one or more patient parameters associated with the new patient, a predicted menopause outcome trajectory associated with the new patient.

3. The computer-implemented method of claim 1, wherein analyzing the historical EMR data associated with the plurality of historical patients to determine one or more patient parameters associated with each historical patient and a historical menopause outcome trajectory associated with each historical patient includes analyzing the historical EMR data using natural language processing (NLP) techniques.

4. The computer-implemented method of claim 1, wherein the patient parameters include one or more of: patient medical condition parameters, patient demographic parameters, or patient lifestyle parameters.

5. The computer-implemented method of claim 4, wherein the patient medical condition parameters include previous or current medical conditions or symptoms associated with the patient, including one or more of: number or frequency of hot flashes, sleep disruption, fatigue, sexual dysfunction, vaginal dryness, presence of menstruation, characteristics of menstruation, instances of amenorrhea, night sweats, urinary symptoms, mood symptoms, brain fog, changes in ability to focus, hair loss, weight gain, perimenopause or menopause status, arthritis, depression, anxiety, breast cancer, osteoporosis, dizziness, vertigo, inflammation, cardiovascular conditions, diabetes, or central nervous system conditions experienced by the patient.

6. The computer-implemented method of claim 4, wherein the patient demographic parameters include one or more of: an age, a location, or a race or ethnicity associated with the patient.

7. The computer-implemented method of claim 4, wherein the patient lifestyle parameters include one or more of: a body mass index, a volume or frequency of alcohol use, a duration or frequency of smoking use, a diet, a number of children, a relationship status, or a stress level associated with the patient.

8. The computer-implemented method of claim 1, wherein the menopause symptoms of the historical menopause outcome trajectory and the predicted menopause trajectory each include one or more of: number or frequency of hot flashes, sleep disruption, fatigue, sexual dysfunction, vaginal dryness, presence of menstruation, characteristics of menstruation, instances of amenorrhea, night sweats, urinary symptoms, mood symptoms, brain fog, changes in ability to focus, hair loss, weight gain, depression, anxiety, dizziness, vertigo, inflammation, or central nervous system conditions experienced by the patient.

9. The computer-implemented method of claim 1, further comprising analyzing the historical EMR data associated with the plurality of historical patients to determine one or more menopause symptom treatments associated with each historical patient; wherein the training dataset includes menopause symptom treatments associated with each historical patient, and wherein training the menopause outcome machine learning model to generate the predicted menopause outcome trajectory for a patient based on patient parameters associated with the patient includes training the menopause outcome machine learning model to generate predicted menopause symptom treatments for alleviating or preventing one or more symptoms of the predicted menopause outcome trajectory.

10. The computer-implemented method of claim 9, wherein the menopause symptom treatments include one or more of: biologics, steroids, nonsteroidal anti-inflammatory drugs (NSAIDs), gabapentin or pregablin, leuperelin, hormone therapy, chemotherapy, metformin, sodium-glucose co-transporter-2 (SGLT2), peroxisome proliferator-activated receptors (PPAR), sulfonylureas, dipeptidyl-peptidase 4 (DPP4), insulin, orlistat, synthetic thyroid, statins, glucagon-like peptide-1 (GLP-1), angiotensin II receptor blockers (ARBs), calcium channel blockers (CCBs), angiotensin converting enzyme (ACE), diuretics, ambien, selective serotonin reuptake inhibitors (SSRIs), norepinephrine and dopamine reuptake inhibitors (NDRI), monoamine oxidase inhibitors (MAOIs), oral contraceptives, hormone replacement therapy, topical or vaginal hormone creams, natural or herbal remedies, cognitive therapy, exercise, meditation, vaginal lubricants, diet alterations, psychotherapy, vitamin D, or clonidine.

11. A computer system, comprising:
one or more processors; and
a non-transitory program memory communicatively coupled to the one or more processors and storing executable instructions that, when executed by the one or more processors, cause the processors to:
obtain historical electronic medical record (EMR) data associated with a plurality of historical patients;
analyze the historical EMR data associated with the plurality of historical patients to determine one or more patient parameters associated with each historical patient and a historical menopause outcome trajectory associated with each historical patient;
generate a training dataset that includes patient parameters and menopause outcome trajectories associated with each historical patient; and
train a menopause outcome machine learning model, using the training dataset, to generate a predicted menopause outcome trajectory for patients based on patient parameters associated with the patient, wherein the predicted menopause outcome trajectory includes or consists of predictions of one or more of: an age of onset of perimenopause for the patient, an age of onset of menopause for the patient, an age of onset of post-menopause for the patient, particular menopause symptoms experienced by the patient, an age of onset of each menopause symptom experienced by the patient, or a severity level associated with each menopause symptom experienced by the patient.

12. The computer system of claim 11, wherein the executable instructions, when executed by the one or more processors, further cause the processors to:
apply the trained menopause outcome machine learning model to one or more patient parameters associated with a new patient; and
generate, based on applying the trained menopause outcome machine learning model to the one or more patient parameters associated with the new patient, a predicted menopause outcome trajectory associated with the new patient.

13. The computer system of claim 11, wherein the executable instructions, when executed by the one or more processors, cause the processors to analyze the historical EMR data associated with the plurality of historical patients to determine one or more patient parameters associated with each historical patient and a historical menopause outcome trajectory associated with each historical patient using natural language processing (NLP) techniques.

14. The computer system of claim 11, wherein the patient parameters include one or more of: patient medical condition parameters, patient demographic parameters, or patient lifestyle parameters.

15. The computer system of claim 14, wherein the patient medical condition parameters include previous or current medical conditions or symptoms associated with the patient, including one or more of: number or frequency of hot flashes, sleep disruption, fatigue, sexual dysfunction, vaginal dryness, presence of menstruation, characteristics of menstruation, instances of amenorrhea, night sweats, urinary symptoms, mood symptoms, brain fog, changes in ability to focus, hair loss, weight gain, perimenopause or menopause status, arthritis, depression, anxiety, breast cancer, osteoporosis, dizziness, vertigo, inflammation, cardiovascular conditions, diabetes, or central nervous system conditions experienced by the patient.

16. The computer system of claim 14, wherein the wherein the patient demographic parameters include one or more of: an age, a location, or a race or ethnicity associated with the patient.

17. The computer system of claim 14, wherein the patient lifestyle parameters include one or more of: a body mass index, a volume or frequency of alcohol use, a duration or frequency of smoking use, a diet, a number of children, a relationship status, or a stress level associated with the patient.

18. The computer system of claim 11, wherein the menopause symptoms of the historical menopause outcome trajectory and the predicted menopause outcome trajectory each include one or more of: number or frequency of hot flashes, sleep disruption, fatigue, sexual dysfunction, vaginal dryness, presence of menstruation, characteristics of menstruation, instances of amenorrhea, night sweats, urinary symptoms, mood symptoms, brain fog, changes in ability to focus, hair loss, weight gain, depression, anxiety, dizziness, vertigo, inflammation, or central nervous system conditions experienced by the patient.

19. The computer system of claim 11, wherein the executable instructions, when executed by the one or more processors, further cause the processors to analyze the historical EMR data associated with the plurality of historical patients to determine one or more menopause symptom treatments associated with each historical patient; and wherein the training dataset includes menopause symptom treatments associated with each historical patient, and wherein training the menopause outcome machine learning model to generate the predicted menopause outcome trajectory for a patient based on patient parameters associated with the patient includes training the menopause outcome machine learning model to predict menopause symptom treatments for alleviating or preventing one or more menopause symptoms of the predicted menopause outcome trajectory.

20. The computer system of claim 19, wherein the menopause symptom treatments include one or more of: biologics, steroids, nonsteroidal anti-inflammatory drugs (NSAIDs), gabapentin or pregablin, leuperelin, hormone therapy, chemotherapy, metformin, sodium-glucose co-transporter-2 (SGLT2), peroxisome proliferator-activated receptors (PPAR), sulfonylureas, dipeptidyl-peptidase 4 (DPP4), insulin, orlistat, synthetic thyroid, statins, glucagon-like peptide-1 (GLP-1), angiotensin II receptor blockers (ARBs), calcium channel blockers (CCBs), angiotensin converting enzyme (ACE), diuretics, ambien, selective serotonin reuptake inhibitors (SSRIs), norepinephrine and dopamine reuptake inhibitors (NDRI), monoamine oxidase inhibitors (MAOIs), oral contraceptives, hormone replacement therapy, topical or vaginal hormone creams, natural or herbal remedies, cognitive therapy, exercise, meditation, vaginal

21. A non-transitory computer readable storage medium storing computer-readable instructions that, when executed by one or more processors, cause the one or more processors to:
obtain historical electronic medical record (EMR) data associated with a plurality of historical patients;
analyze the historical EMR data associated with the plurality of historical patients to determine one or more patient parameters associated with each historical patient and a historical menopause outcome trajectory associated with each historical patient;
generate a training dataset that includes patient parameters and menopause outcome trajectories associated with each historical patient; and
train a menopause outcome machine learning model, using the training dataset, to generate a predicted menopause outcome trajectory for a patient based on patient parameters associated with the patient, wherein the predicted menopause outcome trajectory includes or consists of one or more of: an age of onset of perimenopause for the patient, an age of onset of menopause for the patient, an age of onset of post-menopause for the patient, particular menopause symptoms experienced by the patient, an age of onset of each menopause symptom experienced by the patient, or a severity level associated with each menopause symptom experienced by the patient.

22. The non-transitory computer readable storage medium of claim 21, wherein the computer-readable instructions, when executed by the one or more processors, further cause the processors to:
apply the trained menopause outcome machine learning model to one or more patient parameters associated with a new patient; and
generate, based on applying the trained menopause outcome machine learning model to the one or more patient parameters associated with the new patient, a predicted menopause outcome trajectory associated with the new patient.

23. The non-transitory computer readable storage medium of claim 21, wherein the computer-readable instructions, when executed by the one or more processors, cause the processors to:
analyze the historical EMR data associated with the plurality of historical patients to determine one or more patient parameters associated with each historical patient and a historical menopause outcome trajectory associated with each historical patient using natural language processing (NLP) techniques.

24. The non-transitory computer readable storage medium of claim 21, wherein the patient parameters include one or more of: patient medical condition parameters, patient demographic parameters, or patient lifestyle parameters.

25. The non-transitory computer readable storage medium of claim 24, wherein the patient medical condition parameters include previous or current medical conditions or symptoms associated with the patient, including one or more of: number or frequency of hot flashes, sleep disruption, fatigue, sexual dysfunction, vaginal dryness, presence of menstruation, characteristics of menstruation, instances of amenorrhea, night sweats, urinary symptoms, mood symptoms, brain fog, changes in ability to focus, hair loss, weight gain, perimenopause or menopause status, arthritis, depression, anxiety, breast cancer, osteoporosis, dizziness, vertigo, inflammation, cardiovascular conditions, diabetes, or central nervous system conditions experienced by the patient.

26. The non-transitory computer readable storage medium of claim 24, wherein the patient demographic parameters include one or more of: an age, a location, or a race or ethnicity associated with the patient.

27. The non-transitory computer readable storage medium of claim 24, wherein the patient lifestyle parameters include one or more of: a body mass index, a volume or frequency of alcohol use, a duration or frequency of smoking use, a diet, a number of children, a relationship status, or a stress level associated with the patient.

28. The non-transitory computer readable storage medium of claim 24, wherein the menopause symptoms of the historical menopause outcome trajectory and the predicted menopause outcome trajectory each include one or more of: number or frequency of hot flashes, sleep disruption, fatigue, sexual dysfunction, vaginal dryness, presence of menstruation, characteristics of menstruation, instances of amenorrhea, night sweats, urinary symptoms, mood symptoms, brain fog, changes in ability to focus, hair loss, weight gain, depression, anxiety, dizziness, vertigo, inflammation, or central nervous system conditions experienced by the patient.

29. The non-transitory computer readable storage medium of claim 21, wherein the computer-readable instructions, when executed by the one or more processors, further cause the processors to analyze the historical EMR data associated with the plurality of historical patients to determine one or more menopause symptom treatments associated with each historical patient; and wherein the training dataset includes menopause symptom treatments associated with each historical patient, and wherein training the menopause outcome machine learning model to generate the predicted menopause outcome trajectory for a patient based on patient parameters includes training the menopause outcome machine learning model to predict menopause symptom treatments for preventing or alleviating one or more menopause symptoms of the predicted menopause outcome trajectory.

30. The non-transitory computer readable storage medium of claim 29, wherein the menopause symptom treatments include one or more of: biologics, steroids, nonsteroidal anti-inflammatory drugs (NSAIDs), gabapentin or pregablin, leuperelin, hormone therapy, chemotherapy, metformin, sodium-glucose co-transporter-2 (SGLT2), peroxisome proliferator-activated receptors (PPAR), sulfonylureas, dipeptidyl-peptidase 4 (DPP4), insulin, orlistat, synthetic thyroid, statins, glucagon-like peptide-1 (GLP-1), angiotensin II receptor blockers (ARBs), calcium channel blockers (CCBs), angiotensin converting enzyme (ACE), diuretics, ambien, selective serotonin reuptake inhibitors (SSRIs), norepinephrine and dopamine reuptake inhibitors (NDRI), monoamine oxidase inhibitors (MAOIs), oral contraceptives, hormone replacement therapy, topical or vaginal hormone creams, natural or herbal remedies, cognitive therapy, exercise, meditation, vaginal lubricants, diet alterations, psychotherapy, vitamin D, or clonidine.

31. A computer-implemented method, comprising:
obtaining, by one or more processors, patient parameters associated with a patient;
analyzing, by the one or more processors, the patient parameters associated with the patient using a trained menopause outcome machine learning model; and
generating, by the one or more processors, using the trained menopause outcome machine learning model, a predicted menopause outcome trajectory for the patient based on the patient parameters associated with the patient, wherein the menopause outcome trajectory consists of or includes one or more of: an age of onset of perimenopause for the patient, an age of onset of menopause for the patient, an age of onset of post-menopause for the patient, particular menopause symptoms experienced by the patient, an age of onset of each menopause symptom experienced by the patient, or a severity level associated with each menopause symptom experienced by the patient; and
wherein the menopause outcome machine learning model is trained by:
obtaining historical electronic medical record (EMR) data associated with a plurality of historical patients;
analyzing the historical EMR data associated with the plurality of historical patients to determine one or more historical patient parameters associated with each historical patient and a historical menopause outcome trajectory associated with each historical patient;
generating a training dataset that includes historical patient parameters and historical menopause outcome trajectories associated with each historical patient; and
training the menopause outcome machine learning model, using the training dataset, to generate a predicted menopause outcome trajectory for new patients based on new patient parameters.

32. A computer system, comprising:
one or more processors; and
a non-transitory program memory communicatively coupled to the one or more processors and storing executable instructions that, when executed by the one or more processors, cause the processors to:
obtain patient parameters associated with a patient;
analyze the patient parameters associated with the patient using a trained menopause outcome machine learning model; and
generate, using the trained menopause outcome machine learning model, a predicted menopause outcome trajectory for the patient based on the patient parameters associated with the patient, wherein the menopause outcome trajectory consists of or includes one or more of: an age of onset of perimenopause for the patient, an age of onset of menopause for the patient, an age of onset of post-menopause for the patient, particular menopause symptoms experienced by the patient, an age of onset of each menopause symptom experienced by the patient, or a severity level associated with each menopause symptom experienced by the patient; and
wherein the menopause outcome machine learning model is trained by:
obtaining historical electronic medical record (EMR) data associated with a plurality of historical patients;
analyzing the historical EMR data associated with the plurality of historical patients to determine one or more historical patient parameters associated with each historical patient and a historical menopause outcome trajectory associated with each historical patient;
generating a training dataset that includes historical patient parameters and historical menopause outcome trajectories associated with each historical patient; and
training the menopause outcome machine learning model, using the training dataset, to generate a predicted menopause outcome trajectory for new patients based on new patient parameters.

33. A non-transitory computer readable storage medium storing computer-readable instructions that, when executed by one or more processors, cause the one or more processors to:
obtain patient parameters associated with a patient;
analyze the patient parameters associated with the patient using a trained menopause outcome machine learning model; and
generate, using the trained menopause outcome machine learning model, a predicted menopause outcome trajectory for the patient based on the patient parameters associated with the patient, wherein the menopause outcome trajectory includes or consists of one or more of: an age of onset of perimenopause for the patient, an age of onset of menopause for the patient, an age of onset of post-menopause for the patient, particular menopause symptoms experienced by the patient, an age of onset of each menopause symptom experienced by the patient, or a severity level associated with each menopause symptom experienced by the patient; and
wherein the menopause outcome machine learning model is trained by:
obtaining historical electronic medical record (EMR) data associated with a plurality of historical patients;
analyzing the historical EMR data associated with the plurality of historical patients to determine one or more historical patient parameters associated with each historical patient and a historical menopause outcome trajectory associated with each historical patient;
generating a training dataset that includes historical patient parameters and historical menopause outcome trajectories associated with each historical patient; and
training the menopause outcome machine learning model, using the training dataset, to generate a predicted menopause outcome trajectory for new patients based on new patient parameters.
